(19) United States
(12) Patent Application Publication
(10) Pub. No.: US 2012/0010553 A1
(43) Pub. Date: Jan. 12, 2012

(54) BLOOD COLLECTING SYSTEM

(75) Inventors: Magnus Alqvist, Helsingborg (SE); Daniel Carlson, Lulea (SE)

(73) Assignee: Ljungberg & Kogel, Helsingborg (SE)

(21) Appl. No.: 12/832,552

(22) Filed: Jul. 8, 2010

(51) Int. Cl. A61M 39/08

(52) U.S. Cl. 604/4.01

(57) ABSTRACT

Embodiments include a blood collecting system comprising a scale, a blood agitating system, a blood bag and a tube for extracting blood from a donor. The collecting system comprises, or is connected to, a processor for controlling the scale, the blood agitating system and the amount of blood being extracted from the donor. The processor is arranged to store one or more formulas for calculating an optimum amount of blood that can be donated. The processor can receive information regarding which formula to be used, the parameters necessary for the chosen formula. The processor is arranged to process the parameters in the chosen formula to calculate the amount of blood to be extracted. The processor is arranged to use the result of the calculation to control the collecting system to extract the calculated amount of blood and stop when the scale has detected that the calculated amount has been extracted.

### TECHNICAL FIELD

The invention relates to a blood collecting system comprising a scale, a blood agitating system, a blood bag and a tube for extracting blood from a donor. The collecting system comprises, or is connected to, a processor for controlling the scale, the blood agitating system and the amount of blood being extracted from the donor. The invention also relates to a method for a system according to the above.

### BACKGROUND ART

Blood donation is an important procedure that saves many lives every year worldwide. Blood donation is almost always voluntary and there are unfortunately not as many donors as the need demands. Therefore, there is mostly a shortage of blood why it is important to be able to collect as much blood as possible from each donor.

Blood is collected in a plastic blood bag connected to a tube and a needle. The blood bag is then also connected to other bags in a so called blood bag system. The blood bag system is used in a post donation process to separate blood components. There are several types of blood bag systems on the market; most of them are designed for a specified volume in ml, e.g. 450 ml or 500 ml. Most blood bags are approved by the relevant regulatory bodies to be filled to ±10% of the listed volume on the label of the collection bag.

During a collection, the blood bag system is placed on a combined scale and mixer that constantly agitates the blood in such way that the blood does not aggregate or coagulate. The combined scale and mixer is hereinafter called “collecting system”. The collecting system is set to a specified volume which is within a range of ±10% of the blood bag volume specified on the collection bag. If for instance a 450 ml blood bag is used, the collecting system is set to collect anywhere from 450 ml−10%, or 445 ml to 450 ml+10%, or 495 ml of blood from a donor. When the scale in the collecting system measures that the intended volume or weight has been reached, an automated clamp will close the tube leading from the donor's arm in order to prevent additional blood from being collected. The volume of blood allowed to be collected to date has been defined by a standard related to a minimum weight of each donor when presenting for donation. The actual allowable collection volume is usually considered to be either 13% or 15% of the total blood volume of the donor. U.S. Pat. No. 5,371,329 teaches a system that takes weight into account according to the above and also relies on blood analysis. A problem with the blood analysis is the cost and time for analyzing the blood and a problem with only relying on weight is that the system becomes too rough regarding input data which leads to restrictions according to the above regarding the amount of blood possible to extract from a donor.

As most blood centers have a minimum weight criteria for donors, they may be deferring donors that could donate blood even though their weight alone does not meet current criteria due to the fact that their total blood volume may be sufficient enough in order to donate the minimum volume of blood necessary for a blood bag. For example: 450 ml−10% or 445 ml. On the other hand, donors may meet the minimum weight criteria and be drawn for the exact bag volume when in essence, based on their total blood volume, they could have donated 450+10% or 495 ml of blood.

There are no good and accurate solutions to this problem today. Most collecting systems are set to a specified volume and will stop collection when that volume is reached. Some collecting system can be set to different volumes; e.g. the collecting system can be set to two different volumes which can be chosen prior to the collection. However, it is quite time consuming to change the volume for each donor so mostly the two volumes are set to mach two different blood bag systems, e.g. 400 ml and 500 ml.

### DISCLOSURE OF INVENTION

In view of prior art there exists a need to automatically approximate an optimum allowable blood volume that each donor may donate for every donor occasion.

The problem is solved by the invention by a collecting system either comprising, or being connected to, a processor that can store one or more formulas for calculating optimum amount of blood that can be donated, and which processor can receive information regarding which formula to be used, the parameters used for the chosen formula and which processor can process the parameters in the chosen formula to calculate the amount of blood to be extracted and then use the result of the calculation to control the collecting system to extract the calculated amount of blood and stop when the scale has detected that the calculated amount has been extracted.

The collecting system may comprise, or be connected to, a display unit that presents the calculated amount of blood so that a user may connect the correct size of blood bag to the system. The display unit may also be arranged to present additional information that could be of use to the user.

The collecting system may comprise, or be connected to, an input unit for use when entering data to the processor. The input unit may be a bar code reader, a key board or a similar device that allows for a user to input data to the collecting system. The input unit may be a freestanding unit being connected by wire to the collecting system or may be connected by a wireless connection to the collecting system. The input unit may also be comprised in the display unit and the display unit may be equipped with a touch sensitive screen for use of at least a part of the screen as the input unit.

The data may be in the form of programming of the formulas, the choice of formula and the parameters. The collecting system may be set into manual mode where the collecting system comprises a control function where the user actively has to permit the collecting system to extract the calculated amount of blood. The display then displays the calculated blood volume to the user who then enters data by either allowing or denying the collecting system to start extracting. The collecting system may also comprise an automatic function that puts the collecting system in a ready mode for starting extracting blood after the process of calculating the calculated blood amount has been finished. The collecting system may comprise a switch that switches between the automatic function and the manual function. The switch is preferably operated by the user.

As it is not possible to know the total blood volume of each donor directly, there exist formulas that are designed to combine data, such as height, weight and gender of the individual donating blood in order to estimate the total blood volume of the donor. These formulas are used in order to determine the total allowable blood volume that the donor can donate, usually not more than 15% of the estimated blood volume.

The main advantage with this solution is that the optimum amount of blood is collected from a donor. This can be compared to other methods, where the blood bag is not filled to the volume it is capable of contain or where donors are deferred due to them not meeting current criteria that does not take into account their blood volume.

The blood volume of a donor can be estimated by using different formulas. There are many formulas available and they are all based on some information about the donor, e.g. height, weight, gender, age, size of person, waist measurement, body fat, blood type, number of days since the last donation, ethnic descent, and other parameters that can be useful dependent on which formulas are programmed into the collecting system for use by choice of the user. These data are fed to the formulas which will calculate an estimated blood volume of the donor. This estimated value is in turn used to calculate how much blood to collect, depending on which blood bag that is used, what percentage of blood is allowable, and how much blood is needed for testing purposes.

Any number of formulas may be implemented in the collecting system, and it is up to the user to choose between the different formulas. Some examples of suitable formulas today are presented below, but the invention is not limited to these formulas because other formulas may become useful dependent on which type of user and which type of situation the collecting system may be used. Important for the invention is however that suitable formulas are programmed into the collecting system in such a way that a user can choose between the formulas, for example, dependent on what parameters are accessible, and/or what type of situation is at hand (for example peace or war situation; trauma situation or a simple collecting and storing situation. In the formulas presented the following abbreviations are used:

w: Weight of the donor in kg
h: Height of the donor in cm
a: Age of the donor in years
e: The natural logarithm

Different formulas are used for male and female donors. Furthermore, more formulas exist and might as well be implemented but are not presented in this document. All the presented formulas are in any form published and thus known from prior art and are not developed in conjunction with this invention.

### Stein Holmes (2008)

Male: ((w^{0.1623}*h^{0.7803})−59.2)*100

Female: ((w^{0.1765}*h^{0.6662})−29.4)*100

### Pearson (1994)

Male: (1486*s)−825+(1578*s)

Female: (1.06*a)+(822*s)+(1395*s)

where s=w^{0.425}*h^{0.725}*0.007184

### Hurley (1975)

Male: (1486*s²)−(4106*s)+4514+(995*e^{0.6085}*^{s})

Female: (1167*s)−479+(1278*s^{1.289})

where s=w^{0.425}*h^{0.725}*0.007184

### Nadler (1962)

Male: (366.9*(h/100)³)+(32.19*w)+604

Female: (356.1*(h/100)³)+(33.08*w)+183.3

### Retzlaff (1969):

Male: (1100*s)+(1630*s)

Female: ((840*s)+(1410*s))

s=((ŵ0.425)*(ĥ0.725)*0.007184)

### Wennesland (1959)

Male: ((1550*s)−890)+((1580*s)−520)

### Female: N/A

s=((ŵ0.425)*(ĥ0.725)*0.007184)

The collecting system needs some information about the donor to be able to estimate the donor blood volume. This information is fed to the collecting system via external interfaces, i.e. a barcode reader, keyboard or similar. The information needed might be different for different formulas.

It is only static data that is needed (weight, height, age, etc.), and no online analysis is needed, e.g. blood analysis. However, blood analysis could be an additional option if a formula is used comprising parameters extracted from blood analysis. All information is equally important; the total blood volume cannot be calculated if one is missing. Some formulas do, however, not require all information, e.g. to date only the Pearson formula uses age as a parameter and only for female donors.

Which formula to use, units of e.g. height and length are all configurable by the user, e.g. when used in the US, feet, inches and pounds can be used for weight and height, when used in EU, cm and kg can be used, etc.

Different formulas might have different inputs, i.e. a formula might be dependent of the age of the donor. When a formula is not depended on a certain data, it is not required to be input to the collecting system.

It is also up to the responsible medical director (or similar) to choose between the different formulas. Different local circumstances might lead to different formulas.

The main advantage with this solution is that an optimum amount of blood is collected from a donor. This can be compared to other methods, where the blood bag is not filled to the volume it is capable of contain or where donors are deferred due to them not meeting current criteria that does not take into account their blood volume. The collecting system according to the invention advantageously comprises a upper and lower limit of blood volume such that the collecting system can refuse, in the automatic mode, or suggest to refuse, in the manual mode, blood to be collected.

Instead of calculating the estimated blood volume inside the collecting system, it is also possible to calculate the volume in an external unit connected to the collecting system. In this case, the donor information is not a demand by the collecting system

The method described in this document can be used on any collecting system which has the capability to receive donor information and/or total volume to collect.

### BRIEF DESCRIPTION OF DRAWINGS

The invention will be described briefly in connection to a number of drawings, where:

FIG. 1 schematically shows a side view of a collecting system according to a first example of the invention;

FIG. 2 schematically shows a side view of a collecting system according to a second example of the invention;

FIG. 3 schematically shows a side view of a collecting system according to a third example of the invention;

FIG. 4 schematically shows a side view of a collecting system according to a fourth example of the invention, and where;

FIG. 5 schematically shows a flow chart of a method according to the invention.

### EMBODIMENT(S) OF THE INVENTION

The invention will be described briefly in connection to a number of drawings, where:

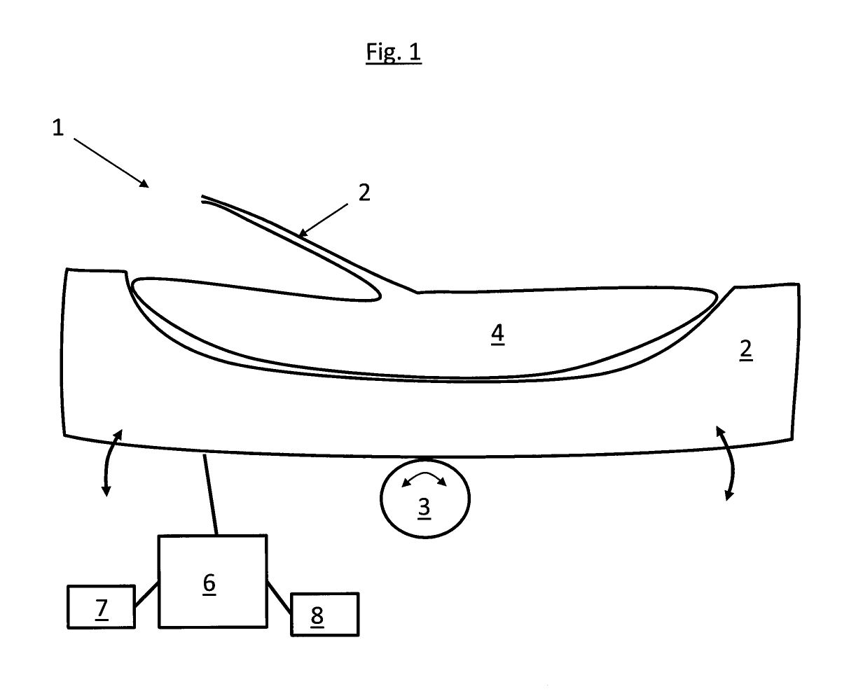

FIG. 1 schematically shows a side view of a blood collecting system 1 according to a first example of the invention. FIG. 1 shows the blood collecting system 1 comprising a scale 2, a blood agitating system 3, a blood bag 4 and a tube 5 for extracting blood from a donor. The collecting system 1 comprises, or is connected to, a processor 6 for controlling the scale 2, the blood agitating system 3 and the amount of blood being extracted from the donor. The processor 6 is arranged to store one or more formulas for calculating an optimum amount of blood that can be donated, and which processor 6 can receive information regarding which formula to be used, the parameters necessary for the chosen formula and which processor 6 is arranged to process the parameters in the chosen formula to calculate the amount of blood to be extracted. The processor 6 is arranged to use the result of the calculation to control the collecting system 1 to extract the calculated amount of blood and stop when the scale 2 has detected that the calculated amount has been extracted.

FIG. 1 shows that the collecting system 1 is connected to a display unit 7 that presents the calculated amount of blood to a user.

FIG. 1 shows that the processor 6 is a freestanding unit being connected by wire to the collecting system 1. However, the processor 6 may be connected by a wireless connection to the collecting system 1.

FIG. 1 shows that the collecting system 1 is connected to an input unit 8 for entering data to the processor 6. The input unit 8 may be a keyboard or a bar code reader or any other suitable means for entering data into the processor in the collecting means. FIG. 1 shows that the input unit 8 is a freestanding unit being connected by wire to the collecting system 1. However, the input unit 8 may be connected by a wireless connection to the collecting system 1. The display unit 7 could be equipped with a touch sensitive screen for use of at least a part of the screen as a keyboard.

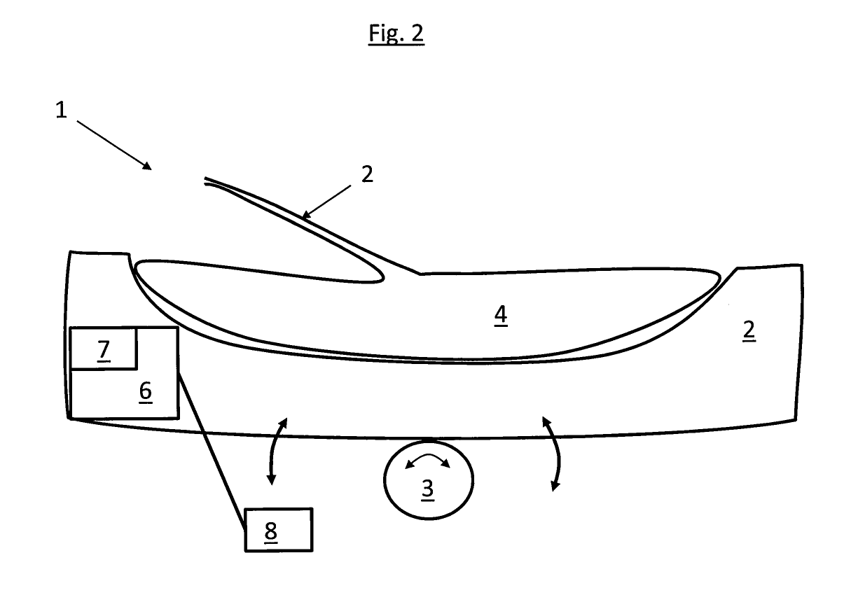

FIG. 2 schematically shows a side view of the collecting system 1 according to a second example of the invention. FIG. 2 shows the same devices as in FIG. 1, but FIG. 2 shows that the collecting system 1 comprises the display unit 7 and the processor 6.

FIG. 2 shows that the input unit 8 is a freestanding unit being connected by wire to the collecting system 1. However, the input unit 8 may be connected by a wireless connection to the collecting system 1. The display unit 7 could be equipped with a touch sensitive screen for use of at least a part of the screen as a keyboard.

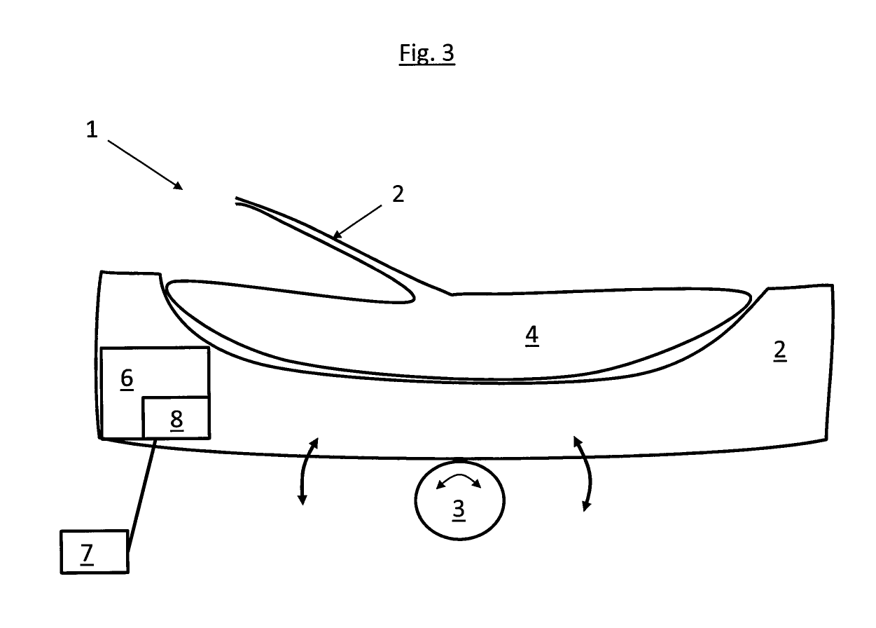

FIG. 3 schematically shows a side view of the collecting system 1 according to a third example of the invention. FIG. 3 shows the same devices as FIGS. 1 and 2, but FIG. 3 shows that the collecting system 1 comprises the input unit 8 and the processor 6.

FIG. 3 shows that the display unit 7 is a freestanding unit being connected by wire to the collecting system 1. However, the display unit 7 may be connected by a wireless connection to the collecting system 1. The display unit 7 could be equipped with a touch sensitive screen for use of at least a part of the screen as a keyboard or bar code reader.

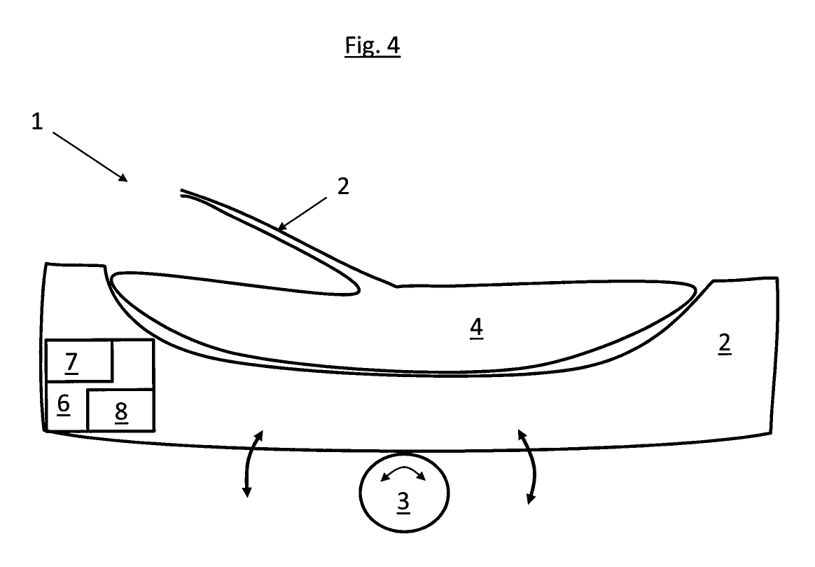

FIG. 4 schematically shows a side view of the collecting system 1 according to a fourth example of the invention. FIG. 4 shows the same devices as FIGS. 1, 2 and 3, but FIG. 4 shows that the collecting system 1 comprises the processor 6, the input unit 8 and the display unit 7.

The display unit 7 could be equipped with a touch sensitive screen for use of at least a part of the screen as a keyboard or bar code reader.

It should be noted that an additional example of the invention, with reference to FIGS. 1-4, would be that the processor 6 is comprised in the collecting system 1, but where the display unit 7 and the input unit 8 would be one or several freestanding units being connected to the processor 6 by wire or by wireless connection.

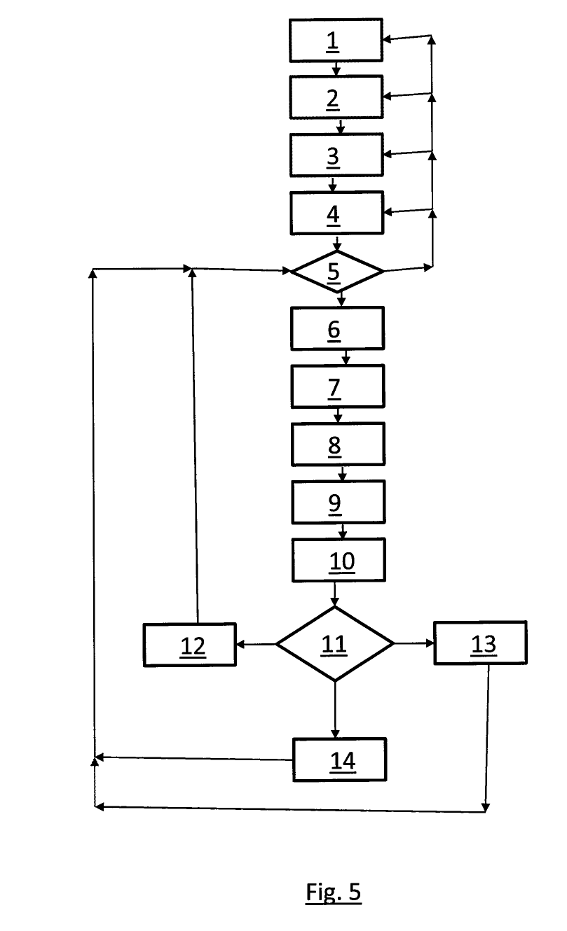

FIG. 5 schematically shows a flow chart of a method according to the invention.

Box 1-4 shows steps during setup of the collecting system.

Box 1: A user inputs which formula to be used. This step can be repeated for changing formula, but if no change is made the chosen formula becomes the set formula used during normal use. The collecting system advantageously comprises a number of formulas that the user can choose from. Goto box 2.

Box 2: The user inputs what sample volume of blood should be used for blood analysis. If no blood analysis is to be done, the user inputs a zero value or a code that disables this function. If the collecting system is not equipped with the possibility to make blood analysis, the step described in box 2 should be omitted from the method. Goto Box 3.

Box 3: The user inputs how many percent of the calculated blood volume to be extracted. This percentage value could be changed by the user at any stage, but remains as a set value unless changed. Goto Box 4.

Box 4: The user defines what unit the processor should use for the parameters and the chosen formulas. Here, units could be kilogram, liters, pounds, gallons etc., dependent on what country you are in or what units the user prefers. Goto Box 5.

Box 5-14 refer to the “normal” use of the collecting system, i.e. after setup of the system.

Box 5: Goto box 1, 2, 3 or 4 to change setup parameters or; input volume of blood bag. The blood bag has a minimum volume and a maximum volume of blood suitable for the bag and these limit values are either input by the user or the processor already has pre-set values with reference to blood bag volume. In the latter case the user only inputs volume of blood bag. Goto Box 6

Box 6: Input gender of donor. Goto Box 7

Box 7: Input weight of donor. Goto Box 8

Box 8: Input height of donor. Goto Box 9

Box 9: Input age of donor. Goto Box 10

Box 10: Input additional parameters to be used by the formulas. Goto Box 11

Box 11: The processor calculates blood volume of donor and uses the percentage value from box 3 to calculate the amount of blood to be extracted. The processor converts blood volume to weight or vice versa for use of the scale for measuring the amount of blood extracted. If the calculated amount is less than the minimum volume the processor will not allow the collecting system to collect blood. The collecting system may then be arranged to display a warning to the user regarding the refusal.
If calculated amount is between the limit values, Goto box 12.
If the processor refuses to collect due to less than minimum value, Goto Box 13.
If the processor refuses to collect due to more than maximum value, Goto Box 14.

Box 12: The processor starts collecting the calculated amount of blood and stops when the scale has detected the correct amount. Goto box 5

Box 13: The processor refuses to collect blood, displays this for the user and returns to ready mode. Goto box 5.

Box 14: If the calculated amount exceeds the maximum volume, the processor may be arranged to refuse the collecting system to collect blood until a user inputs an order to proceed. If the processor refuses to collect blood this is advantageously displayed for the user that can assess the situation and give the order to proceed or abort. The process of giving the user the possibility to control the collecting system dependent on information from the processor has previously been referred to as a manual mode. The collecting system may also be arranged for use in an automatic mode where the processor controls the collecting means dependent on pre-set method steps. In the automatic mode: If the calculated amount exceeds the maximum volume, the processor may be arranged to automatically proceed by collecting the maximum volume and stop when the scale has detected the correct amount collected. Goto box 5.

The method could comprise the step of setting the collecting system into manual mode where the collecting system comprises a control function where the user actively permits the collecting system to extract the calculated amount of blood after the processor has calculated the amount to be extracted; or setting the collecting system into an automatic function that puts the collecting system in a ready mode for starting extracting blood after the process of calculating the calculated blood amount has been finished. The invention is not limited to such a switch possibility, but the collecting system may be pre-set into a manual type of mode or an automatic type of mode according to what has been described. Hence, in the pre-set type of collecting system the user does not have to choose between manual or automatic mode.

At present, the collecting system and the method uses the following formula for calculating the amount of blood to be extracted, but additional formulas may be added dependent on future findings within the field:

Male: ((w^{0.1623}*h^{0.7803})−59.2)*100

Female: ((w^{0.1765}*h^{0.6662})−29.4)*100 Formula 1

Male: (1486*s)−825+(1578*s)

Female: (1.06*a)+(822*s)+(1395*s)

where s=w^{0.425}*h^{0.725}*0.007184 Formula 2

Male: (1486*s²)−(4106*s)+4514+(995*e^{0.6085}*^{s})

Female: (1167*s)−479+(1278*s^{1.289})

where s=w^{0.425}*h^{0.725}*0.007184 Formula 3

Male: (366.9*(h/100)³)+(32.19*w)+604

Female: (356.1*(h/100)³)+(33.08*w)+183.3 Formula 4

Male: (1100*s)+(1630*s)

Female: ((840*s)+(1410*s))

s=((ŵ0.425)*(ĥ0.725)*0.007184) Formula 5

Male: ((1550*s)−890)+((1580*s)−520)

Female: N/A

s=((ŵ0.425)*(ĥ0.725)*0.007184) Formula 6

wherein the parameters are;
w: Weight of the donor in kg
h: Height of the donor in cm
a: Age of the donor in years
e: The natural logarithm

What is claimed is:

1. A blood collecting system comprising:
a scale,
a blood agitating system,
a blood bag and
a tube for extracting blood from a donor,
a processor included in or coupled to the collecting system,
the processor for controlling the scale, the blood agitating system and the amount of blood being extracted from the donor,
wherein the processor is arranged to store one or more formulas for calculating an optimum amount of blood that can be donated, and which processor is arranged to receive information regarding which formula to be used, the parameters necessary for the chosen formula,
wherein the processor is arranged to process the parameters in the chosen formula to calculate the amount of blood to be extracted,
wherein the processor is arranged to use the result of the calculation to control the collecting system to extract the calculated amount of blood and stop when the scale has detected that the calculated amount has been extracted.

2. The collecting system according to claim 1, wherein the collecting system comprises, or is connected to, a display unit that presents the calculated amount of blood to a user.

3. The collecting system according to claim 1, wherein the collecting system comprises, or is connected to, an input unit for entering data to the processor.

4. The collecting system according to claim 3, wherein the input unit is a freestanding unit being connected by wire to the collecting system or is connected by a wireless connection to the collecting system; or wherein the input unit is comprised in the display unit and wherein the display unit is equipped with a touch sensitive screen for use of at least a part of the screen as the input unit.

5. The collecting system according to claim 1, wherein the collecting system is arranged to be set into manual mode where the collecting system comprises a control function where the user actively has to permit the collecting system to extract the calculated amount of blood after the processor has calculated the amount to be extracted; or arranged to be set into an automatic function that puts the collecting system in a ready mode for starting extracting blood after the process of calculating the calculated blood amount has been finished.

6. The collecting system according to claim 5, wherein the collecting system comprises a switch that switched between the automatic function and the manual function.

7. The collecting system according to claim 1, wherein one or more of the following formulas are used for calculating amount of blood in the donor:
Formula 1:
Male: ((w^{0.1623}*h^{0.7803})−59.2)*100
Female: ((w^{0.1765}*h^{0.6662})−29.4)*100
Formula 2:
Male: (1486*s)−825+(1578*s)
Female: (1.06*a)+(822*s)+(1395*s)
where s=w^{0.425}*h^{0.725}*0.007184
Formula 3:
Male: (1486*s²)−(4106*s)+4514+(995*e^{0.6085}*^{s})
Female: (1167*s)−479+(1278*s^{1.289})
where s=w^{0.425}*h^{0.725}*0.007184
Formula 4:
Male: (366.9*(h/100)³)+(32.19*w)+604
Female: (356.1*(h/100)³)+(33.08*w)+183.3
Formula 5:
Male: (1100*s)+(1630*s)
Female: ((840*s)+(1410*s))
s=((ŵ0.425)*(ĥ0.725)*0.007184)
Formula 6:
Male: ((1550*s)−890)+((1580*s)−520)
Female: N/A
s=((ŵ0.425)*(ĥ0.725)*0.007184)
wherein the parameters are;
w: Weight of the donor in kg
h: Height of the donor in cm
a: Age of the donor in years
e: The natural logarithm.

8. A method for a blood collecting system comprising a scale, a blood agitating system, a blood bag and a tube for extracting blood from a donor, a processor included in or coupled to the collecting system, the processor for controlling the scale, the blood agitating system and the amount of blood being extracted from the donor,
wherein the processor is arranged to store one or more formulas and that the calculates an optimum amount of blood that can be donated by use of the formulas, and which processor receives information regarding which formula to be used, the parameters necessary for the chosen formula and which processor processes the parameters in the chosen formula and calculates the amount of blood to be extracted, wherein the processor uses the result of the calculation to control the collecting system to extract the calculated amount of blood and stop when the scale has detected that the calculated amount has been extracted.

9. The method according to claim 8, wherein the collecting system comprises, or is connected to, a display unit that presents the calculated amount of blood to a user.

10. The method according to claim 8, wherein the collecting system comprises, or is connected to, an input unit for entering data to the processor.

11. The method according to claim 10, wherein the input unit is a freestanding unit being connected by wire to the collecting system or is connected by a wireless connection to the collecting system; or wherein the input unit is comprised in the display unit and wherein the display unit is equipped with a touch sensitive screen for use of at least a part of the screen as the input unit.

12. The method according to claim 8, wherein the method comprises setting the collecting system into manual mode where the collecting system comprises a control function where the user actively permits the collecting system to extract the calculated amount of blood after the processor has calculated the amount to be extracted; or setting the collecting system into an automatic function that puts the collecting system in a ready mode for starting extracting blood after the process of calculating the calculated blood amount has been finished.

13. The method according to claim 8, wherein one or more of the following formulas are used for calculating the amount of blood in the donor:
Formula 1:
Male: ((w^{0.1623}*h^{0.7803})−59.2)*100
Female: ((w^{0.1765}*h^{0.6662})−29.4)*100
Formula 2:
Male: (1486*s)−825+(1578*s)
Female: (1.06*a)+(822*s)+(1395*s)
where s=w^{0.425}*h^{0.725}*0.007184
Formula 3:
Male: (1486*s²)−(4106*s)+4514+(995*e^{0.6085}*^{s})
Female: (1167*s)−479+(1278*s^{1.289})
where s=w^{0.425}*h^{0.725}*0.007184
Formula 4:
Male: (366.9*(h/100)³)+(32.19*w)+604
Female: (356.1*(h/100)³)+(33.08*w)+183.3
Formula 5:
Male: (1100*s)+(1630*s)
Female: ((840*s)+(1410*s))
s=((ŵ0.425)*(ĥ0.725)*0.007184)
Formula 6:
Male: ((1550*s)−890)+((1580*s)−520)
Female: N/A
s=((ŵ0.425)*(ĥ0.725)*0.007184)
wherein the parameters are;
w: Weight of the donor in kg
h: Height of the donor in cm
a: Age of the donor in years
e: The natural logarithm.

14. The method according to claim 8, wherein the method comprises the user setting a percentage of the donor blood volume as the calculated amount of blood to be extracted.
